(19) United States
(12) Patent Application Publication
(10) Pub. No.: US 2025/0140087 A1
(43) Pub. Date: May 1, 2025

(54) TACTILE SENSATION PRESENTING DEVICE

(71) Applicant: Sharp Display Technology Corporation, Kameyama City (JP)

(72) Inventors: Masafumi UENO, Kameyama City (JP); Tomonori YOSHIDA, Kameyama City (JP); Takuma YAMAMOTO, Kameyama City (JP); Yasuhiro SUGITA, Kameyama City (JP)

(21) Appl. No.: 18/910,229

(22) Filed: Oct. 9, 2024

(30) Foreign Application Priority Data
Oct. 30, 2023 (JP) 2023-185367

(51) Int. Cl. G08B 6/00; A61N 1/04

(52) U.S. Cl. CPC G08B 6/00; A61N 1/0476

(57) ABSTRACT

A tactile sensation presenting device includes: a tactile sensation presenting section having a plurality of electrodes to present a tactile sensation by electrical stimulation at a specific portion of a user; and a control section capable of controlling the tactile sensation presenting section to provide electrical stimulation using at least one of the plurality of electrodes as an anode and at least another one of the plurality of electrodes as a cathode. The control section measures a flowability of an electric current through the specific portion of the user and adjusts an execution mode of the electrical stimulation based on a result of the measurement.

### BACKGROUND

### 1. Technical Field

The present invention relates to tactile sensation presenting devices and particularly to tactile sensation presenting devices configured to present tactile sensations by electrical stimulation.

### 2. Description of the Related Art

In recent years, tactile sensation presenting devices capable of presenting tactile sensations to a user (also referred to as “haptics devices”) have been receiving attention and have already been applied to a variety of uses, such as medical, educational, entertainment, and remote operation uses. Several types of tactile sensation presenting devices have been known. The tactile sensation presenting devices most widely used nowadays are force application type tactile sensation presenting devices that are configured to present tactile sensations by applying force to a user and vibration type tactile sensation presenting devices that are configured to present tactile sensations by transmitting vibrations to a user. Recently, electrical stimulation type tactile sensation presenting devices that are configured to present tactile sensations by electrical stimulation are promising. An electrical stimulation type tactile sensation presenting device is disclosed in, for example, Japanese Laid-Open Patent Publication No. 2006-251948.

In the electrical stimulation type tactile sensation presenting device, a plurality of electrodes are arrayed with a predetermined pitch in a portion that is to come into contact with user's skin, and current paths from anodes to cathodes are formed under the skin, whereby sensory receptors under the skin (e.g., Meissner's corpuscles) are stimulated to present tactile sensations.

### SUMMARY

The flowability of an electric current through a portion near the surface (skin) of the human body varies depending on the individual difference in internal resistance unique to each individual and the environment in which the device is used. Therefore, it is difficult for electrical stimulation type tactile sensation presenting devices to present accurate tactile sensations while ensuring adequate safety. If an excessive electric current flows, it can be hazardous. On the other hand, if it is difficult for the electric current to flow, tactile sensations may not be felt at all. This is because, even if an electric current flows at the surface of the human body, it is not felt as a tactile sensation before the electric current reaches the sensory receptors that sense tactile sensations.

Embodiments of the present invention were conceived in view of the above-described problems and are directed to providing tactile sensation presenting devices that are capable of presenting accurate tactile sensations while ensuring adequate safety.

This specification discloses tactile sensation presenting devices described in the following items.

### [Item 1]

A tactile sensation presenting device including:
a tactile sensation presenting section having a plurality of electrodes to present a tactile sensation by electrical stimulation at a specific portion of a user; and
a control section capable of controlling the tactile sensation presenting section to provide electrical stimulation using at least one of the plurality of electrodes as an anode and at least another one of the plurality of electrodes as a cathode,
wherein the control section measures a flowability of an electric current through the portion and adjusts an execution mode of the electrical stimulation based on a result of the measurement.

### [Item 2]

The tactile sensation presenting device of Item 1, wherein
the tactile sensation presenting section further includes a plurality of measurement terminals, and
the control section performs the measurement using the plurality of measurement terminals.

### [Item 3]

The tactile sensation presenting device of Item 2, wherein the plurality of measurement terminals include a first terminal and a second terminal, the first terminal and the second terminal having different areas as viewed in plan.

### [Item 4]

The tactile sensation presenting device of Item 3, wherein
a distance from a center of the tactile sensation presenting section to the second terminal is greater than a distance from the center to the first terminal as viewed in plan, and
the area of the second terminal as viewed in plan is greater than the area of the first terminal as viewed in plan.

### [Item 5]

The tactile sensation presenting device of Item 1, wherein the control section performs the measurement using at least two of the plurality of electrodes.

### [Item 6]

The tactile sensation presenting device of Item 5, wherein the number of electrodes which function as positive terminals in the measurement and the number of electrodes which function as negative terminals in the measurement are each two or more.

### [Item 7]

The tactile sensation presenting device of Item 5 or 6, wherein the number of electrodes which function as positive terminals in the measurement and the number of electrodes which function as negative terminals in the measurement are different from each other.

### [Item 8]

The tactile sensation presenting device of any of Items 1 to 7 wherein, in adjusting the execution mode of the electrical stimulation, the control section adjusts a distance between the anode and the cathode.

### [Item 9]

The tactile sensation presenting device of any of Items 1 to 8 wherein, in adjusting the execution mode of the electrical stimulation, the control section adjusts a total area of the electrode which functions as the anode and a total area of the electrode which functions as the cathode.

### [Item 10]

The tactile sensation presenting device of Item 9 wherein, in adjusting the execution mode of the electrical stimulation, the control section adjusts a ratio between the total area of the electrode which functions as the anode and the total area of the electrode which functions as the cathode.

### [Item 11]

The tactile sensation presenting device of any of Items 1 to 10 wherein, in adjusting the execution mode of the electrical stimulation, the control section adjusts a pattern of a voltage applied to the electrode which functions as the anode and the electrode which functions as the cathode.

### [Item 12]

The tactile sensation presenting device of Item 11 wherein, in adjusting the execution mode of the electrical stimulation, the control section adjusts an application duration of the voltage applied to the electrode which functions as the anode and the electrode which functions as the cathode.

### [Item 13]

The tactile sensation presenting device of Item 11 or 12 wherein, in adjusting the execution mode of the electrical stimulation, the control section adjusts a frequency of the voltage applied to the electrode which functions as the anode and the electrode which functions as the cathode.

### [Item 14]

The tactile sensation presenting device of any of Items 1 to 13, wherein the plurality of electrodes are arrayed in a matrix including a plurality of rows and a plurality of columns.

### [Item 15]

The tactile sensation presenting device of any of Items 1 to 14, wherein
the plurality of electrodes include a plurality of anodic electrodes and a plurality of cathodic electrodes,
the control section is capable of independently switching each of the plurality of anodic electrodes between a state where the anodic electrode is supplied with an anodic potential and a state where the anodic electrode is supplied with a floating potential, and
the control section is capable of independently switching each of the plurality of cathodic electrodes between a state where the cathodic electrode is supplied with a cathodic potential and a state where the cathodic electrode is supplied with a floating potential.

### [Item 16]

The tactile sensation presenting device of Item 15, wherein each of the plurality of cathodic electrodes has such a shape that surrounds at least one of the plurality of anodic electrodes as viewed in plan.

### [Item 17]

The tactile sensation presenting device of any of Items 1 to 16, wherein the specific portion is a fingertip inner portion of the user.

According to embodiments of the present invention, tactile sensation presenting devices can be provided which are capable of presenting accurate tactile sensations while ensuring adequate safety.

### BRIEF DESCRIPTION OF THE DRAWINGS

FIG. 1 is a block diagram schematically showing a VR tactile sensation feedback system 200 that includes a tactile sensation presenting device 100 according to an embodiment of the present invention.

FIG. 2 is diagram for illustrating a fingertip inner portion fp.

FIG. 3 is a plan view schematically showing a tactile sensation presenting section 10 of the tactile sensation presenting device 100.

FIG. 4 is a cross-sectional view schematically showing the tactile sensation presenting section 10.

FIG. 5 is a perspective view schematically showing the tactile sensation presenting section 10.

FIG. 6 illustrates an example of voltage setting in the tactile sensation presenting section 10 in presenting a tactile sensation by electrical stimulation.

FIG. 7 is a plan view schematically showing the tactile sensation presenting section 10.

FIG. 8 is a graph showing an example of the pattern of a voltage applied to a plurality of electrodes 12 of the tactile sensation presenting section 10.

FIG. 9 is a plan view showing an example of a specific configuration for measuring the flowability of an electric current.

FIG. 10 is a cross-sectional view showing an example of a specific configuration for measuring the flowability of an electric current.

FIG. 11 is a plan view showing an example of a specific configuration for measuring the flowability of an electric current.

FIG. 12 is a cross-sectional view showing an example of a specific configuration for measuring the flowability of an electric current.

FIG. 13 is a plan view showing an example of a specific configuration for measuring the flowability of an electric current.

FIG. 14 is a cross-sectional view showing an example of a specific configuration for measuring the flowability of an electric current.

FIG. 15 is a plan view showing an example of a specific configuration for measuring the flowability of an electric current.

FIG. 16 is a plan view showing an example of a specific configuration for measuring the flowability of an electric current.

FIG. 17 is a cross-sectional view showing an example of a specific configuration for measuring the flowability of an electric current.

FIG. 18 illustrates a setting example of an anode 12A and a cathode 12C.

FIG. 19 illustrates a setting example of an anode 12A and cathodes 12C.

FIG. 20 is a graph showing the relationship between the resistance value and the anode-cathode distance d_{AC} in a case where the anode-cathode distance d_{AC} is adjusted according to the measured resistance value.

FIG. 21 is a graph showing the relationship between the anode-cathode distance d_{AC} and the resistance value.

FIG. 22 illustrates a setting example of anodes 12A and cathodes 12C.

FIG. 23 illustrates a setting example of an anode 12A and cathodes 12C.

FIG. 24 is a graph showing the relationship between the resistance value and the number of anodes 12A and the number of cathodes 12C in a case where the number of anodes 12A and the number of cathodes 12C are adjusted according to the measured resistance value.

FIG. 25 is a graph showing the relationship between the resistance value and the number of anodes 12A and the number of cathodes 12C in a case where the ratio between the number of anodes 12A and the number of cathodes 12C is adjusted according to the measured resistance value.

FIG. 26 illustrates a setting example of anodes 12A and cathodes 12C.

FIG. 27 shows examples of the pattern of a voltage applied to electrodes 12 which function as anodes 12A and electrodes 12 which function as cathodes 12C.

FIG. 28 shows examples of the pattern of a voltage applied to electrodes 12 which function as anodes 12A and electrodes 12 which function as cathodes 12C.

FIG. 29 is a graph showing the relationship between the resistance value and the application duration (duty ratio) of the voltage in a case where the application duration of the voltage is adjusted according to the measured resistance value.

FIG. 30 is a graph showing the relationship between the resistance value and the frequency (Hz) of the voltage in a case where the frequency of the voltage is adjusted according to the measured resistance value.

FIG. 31 is a plan view schematically showing the tactile sensation presenting section 10.

FIG. 32 illustrates a setting example of anodes 12A and cathodes 12C.

FIG. 33 illustrates a setting example of anodes 12A and cathodes 12C.

FIG. 34 illustrates a setting example of anodes 12A and cathodes 12C.

### DETAILED DESCRIPTION

Hereinafter, embodiments of the present invention are described with reference to the drawings. In examples described below, a tactile sensation presenting device is used in a VR (Virtual Reality) tactile sensation feedback system, although the use of the tactile sensation presenting device is not limited to such examples.

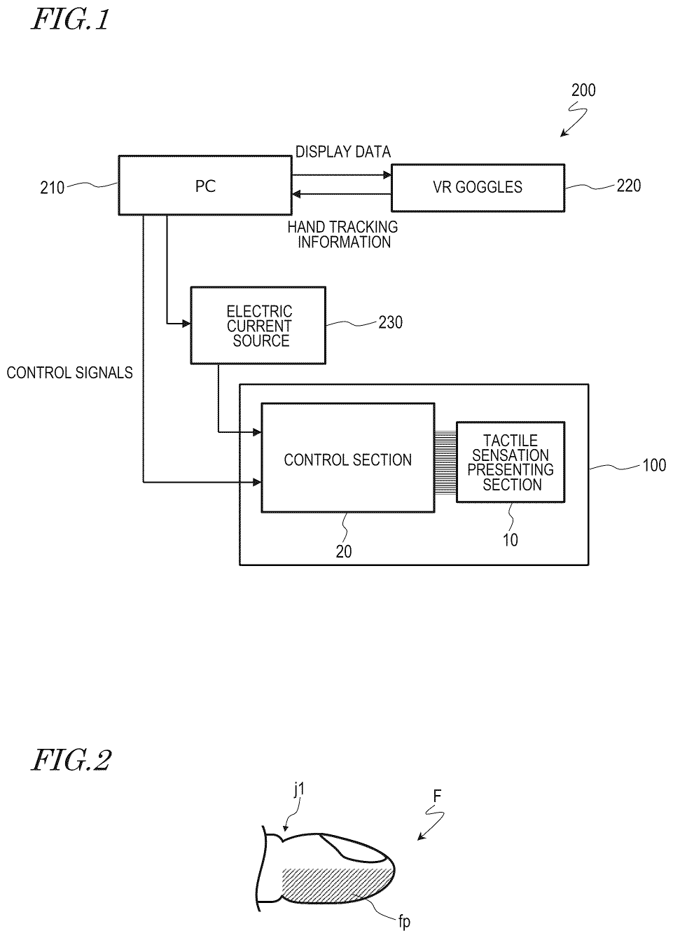

A tactile sensation presenting device 100 of the present embodiment is described with reference to FIG. 1. FIG. 1 is a block diagram schematically showing a VR tactile sensation feedback system (hereinafter, also simply referred to as “system”) 200 that includes a tactile sensation presenting device 100.

The system 200 includes the tactile sensation presenting device 100, a personal computer (PC) 210, VR goggles 220, and an electric current source 230 as shown in FIG. 1.

The tactile sensation presenting device 100 includes a tactile sensation presenting section 10 and a control section 20 for controlling the tactile sensation presenting section 10. The tactile sensation presenting section 10 includes a plurality of electrodes, as will be described later, to present tactile sensations by electrical stimulation at a specific portion of a user. Herein, when the tactile sensation presenting device 100 is used, the tactile sensation presenting section 10 is provided so as to be in contact with the fingertips of a user's hand and presents tactile sensations to the “fingertip inner portion” by electrical stimulation. The “fingertip inner portion” refers to a part fp of the finger F which is located beyond the first joint (distal joint) j1 of the finger F and which is located on the palm side relative to the center when the finger F is viewed from the side as shown in FIG. 2.

In the shown example, the tactile sensation presenting device 100 includes a single tactile sensation presenting section 10, although the number of tactile sensation presenting sections 10 is not limited to one but may be two or more. For example, if the tactile sensation presenting device 100 includes five tactile sensation presenting sections 10, tactile sensations can be presented at each of five fingers F of a user's hand.

The control section 20 controls the tactile sensation presenting section 10. The control section 20 controls the tactile sensation presenting section 10 based on control signals received from the PC 210 in which VR applications are installed. The data transmission between the control section 20 and the PC 210 may be realized by wireless communication or wired communication. The wireless communication and wired communication can be established in compliance with various known communication standards. The control section 20 can be, for example, a control board in which a microcomputer is incorporated.

The tactile sensation presenting section 10 is wired using flexible wires, or the like, so as not to obstruct the movement of the user's hand. The control section 20 can be provided at, for example, a portion corresponding to a user's arm. The tactile sensation presenting section 10 and the control section 20 may be integrated in the form of a glove.

The PC 210 outputs display data to the VR goggles 220, and the VR goggles 220 displays images based on the received display data. The VR goggles 220 also outputs hand tracking information, which is information about the position and movement of the user's hand, to the PC 210. The data transmission between the PC 210 and the VR goggles 220 may be realized by wireless communication or wired communication.

The PC 210 controls the electric current source 230 and the control section 20 via the VR applications in order to present tactile sensations at the fingertip inner portion fp in conjunction with the hand tracking information. The control section 20 controls the operation such that a voltage is selectively applied to specific ones of the plurality of electrodes of the tactile sensation presenting section 10 (i.e., an electric current flows). The electric current source 230 supplies an electric current which is to flow through the electrodes to the tactile sensation presenting section 10 via the control section 20. The electric current source 230 can vary the electric current value for each selected electrode.

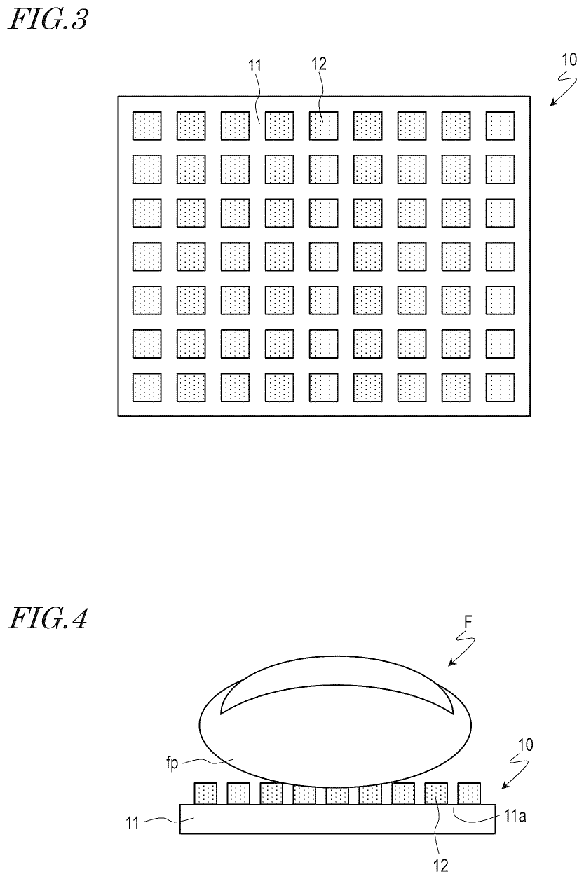

A specific configuration of the tactile sensation presenting section 10 is described with reference to FIG. 3, FIG. 4 and FIG. 5. FIG. 3, FIG. 4 and FIG. 5 are, respectively, a plan view, a cross-sectional view and a perspective view schematically showing the tactile sensation presenting section 10. FIG. 4 and FIG. 5 show a state where the tactile sensation presenting section 10 is in contact with the fingertip inner portion fp.

In the example shown in FIG. 3, the tactile sensation presenting section 10 has a generally rectangular shape as viewed in plan. The ratio between the transverse width (transverse dimension) and the longitudinal width (longitudinal dimension) of the tactile sensation presenting section 10 is not particularly limited. The shape of the tactile sensation presenting sections 10 is not limited to generally rectangular shapes.

The tactile sensation presenting section 10 includes a substrate (support) 11 having a major surface 11a and a plurality of electrodes 12 as shown in FIG. 3, FIG. 4 and FIG. 5.

The substrate 11 may have flexibility. If the substrate 11 has flexibility, the tactile sensation presenting section 10 can be deformed along a rounded part of the fingertips.

The plurality of electrodes 12 are provided on the major surface 11a of the substrate 11. In the example shown in FIG. 3, FIG. 4 and FIG. 5, the plurality of electrodes 12 are arrayed in a matrix including a plurality of rows and a plurality of columns. Herein, each of the electrodes 12 has a generally rectangular shape, and the electrodes 12 have generally equal areas as viewed in plan. Note that, although not shown herein, the tactile sensation presenting section 10 further includes wires connected with the plurality of electrodes 12 and relevant components.

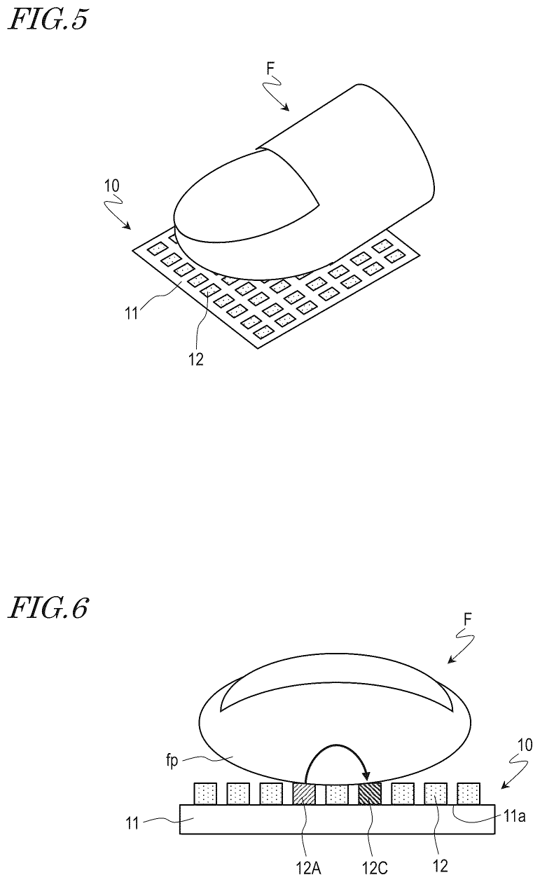

The control section 20 controls the tactile sensation presenting section 10 so as to provide electrical stimulation using at least one of the plurality of electrodes 12 as an anode and at least another one of the plurality of electrodes 12 as a cathode. FIG. 6 illustrates an example of voltage setting in the tactile sensation presenting section 10 in presenting a tactile sensation by electrical stimulation. In presenting a tactile sensation, at least one of the plurality of electrodes 12 is supplied with a predetermined anodic potential so as to function as an anode 12A, and at least another one of the plurality of electrodes 12 is supplied with a predetermined cathodic potential, which is lower than the anodic potential, so as to function as a cathode 12C. Meanwhile, the other electrodes 12 are supplied with a floating potential. When a finger F comes into contact with the tactile sensation presenting section 10 in this state, an electric current flows from the anode 12A to the cathode 12C via the finger F. When the electric current reaches sensory receptors under the skin, it is perceived as a tactile sensation. The pressure (strength of tactile sensations) can be adjusted by changing the magnitude of the flowing electric current, and minute electric current patterns can cause the user to sense a coarse texture.

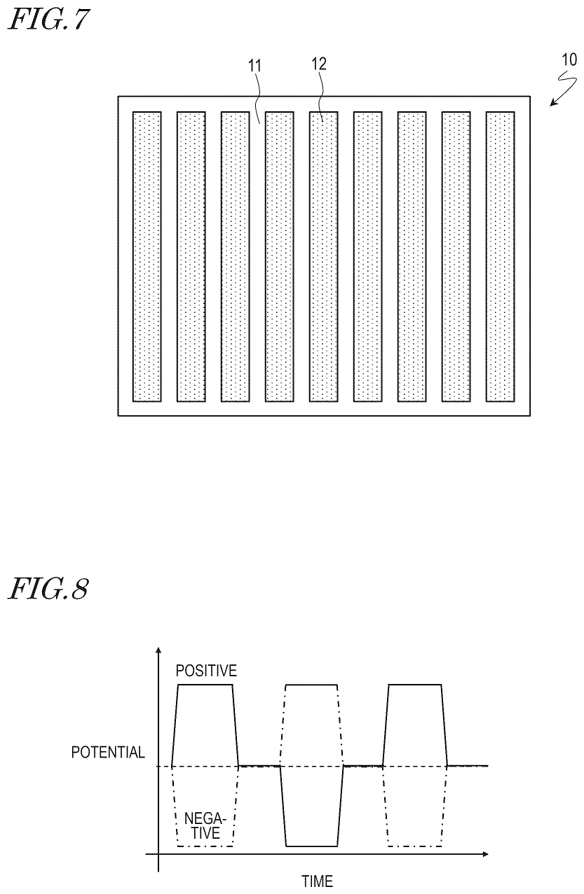

In the example shown in FIG. 3, FIG. 4 and FIG. 5, the plurality of electrodes 12 are arrayed in a matrix, although the array of the electrodes 12 is not limited to this example. FIG. 7 shows another example of the array of the electrodes 12. In the example shown in FIG. 7, the electrodes 12 are in the shape of stripes elongated in the column direction, and the plurality of electrodes 12 are arrayed in a single row and a plurality of columns. Although not shown herein, the electrodes 12 may be in the shape of stripes elongated in the row direction, and the plurality of electrodes 12 may be arrayed in a plurality of rows and a single column.

The pattern of the voltage applied to the electrodes 12 is not limited to such a pattern that some electrodes 12 are always supplied with the anodic potential (e.g., positive potential) while some other electrodes 12 are always supplied with the cathodic potential (e.g., negative potential). FIG. 8 shows another example of the voltage pattern. In FIG. 8, the potential applied to some electrodes 12 is shown by a solid line, and the potential applied to some other electrodes 12 is shown by a chain line. In the example shown in FIG. 8, some electrodes 12 and some other electrodes 12 are supplied with the positive potential and the negative potential alternately with a predetermined period. The electrodes 12 supplied with the positive potential function as the anodes 12A, and the electrodes 12 supplied with the negative potential function as the cathodes 12C. Every time the polarity (positive/negative) of the potential is reversed, the direction of the electric current is also reversed.

The type of tactile sensations to be presented can be defined by the voltage pattern. In the case of the voltage pattern such as shown in FIG. 8, the voltage pattern is determined by the amplitude and frequency (polarity reversal periods) of the voltage. Also, the tactile sensations can be varied by changing the position of the electrodes 12 to which the anodic potential and the cathodic potential are applied (voltage application position) in the tactile sensation presenting section 10. For example, high-resolution tactile sensations can be presented by shifting the voltage application position left and right or up and down within a relatively small region. The tactile sensations can also be varied by changing the distance or velocity of the position shift, the number of repetitions of the position shift, or the like.

The tactile sensation presenting device 100 that includes the tactile sensation presenting section 10 and the control section 20 such as described above can control the position and intensity of tactile sensation stimulation at the fingertips with high resolutions and can reproduce the tactile sensation of tracing an object with fingertips. Therefore, the VR tactile sensation feedback system 200 can be provided which is capable of producing more realistic touch and texture.

The flowability of the electric current in the fingertip inner portion fp varies depending on the individual difference in internal resistance unique to each individual and the environment in which the device is used (e.g., ambient temperature and humidity, degree of dryness of the fingertip inner portion fp). The tactile sensation presenting device 100 of the present embodiment has a configuration which will be described below and is thus capable of presenting accurate tactile sensations while ensuring adequate safety.

In the tactile sensation presenting device 100 according to an embodiment of the present invention, the control section 20 measures the flowability of an electric current through a specific portion (herein, the fingertip inner portion fp) and adjusts the execution mode of electrical stimulation based on the results of the measurement. Therefore, the tactile sensation presenting device 100 can present accurate tactile sensations irrespective of the individual difference in internal resistance or the environment in which the device is used. Hereinafter, measurement of the flowability of the electric current and adjustment of the execution mode of electrical stimulation are specifically described.

### [Measurement of Flowability of Electric Current]

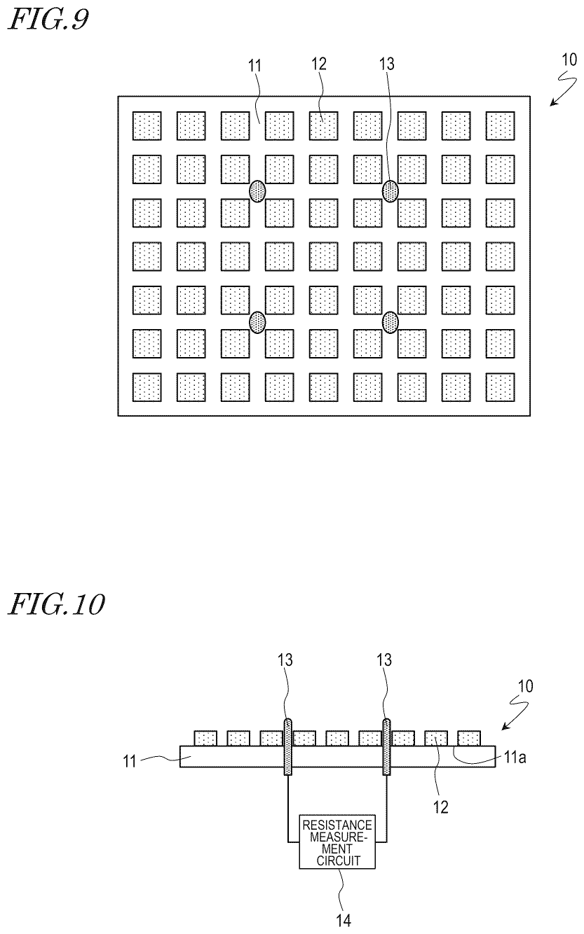

An example of the specific configuration for measuring the flowability of the electric current is described with reference to FIG. 9 and FIG. 10.

In the example shown in FIG. 9 and FIG. 10, the tactile sensation presenting section 10 includes a plurality of measurement terminals 13. These measurement terminals 13 are provided among the electrodes 12 and electrically coupled with a resistance measurement circuit 14. The control section 20 measures the resistance value using the plurality of measurement terminals 13.

In the example described herein, the tactile sensation presenting section 10 includes four measurement terminals 13, although the number of measurement terminals 13 is not limited to four but may be two or more. The arrangement of the measurement terminals 13 is also not limited to the shown example.

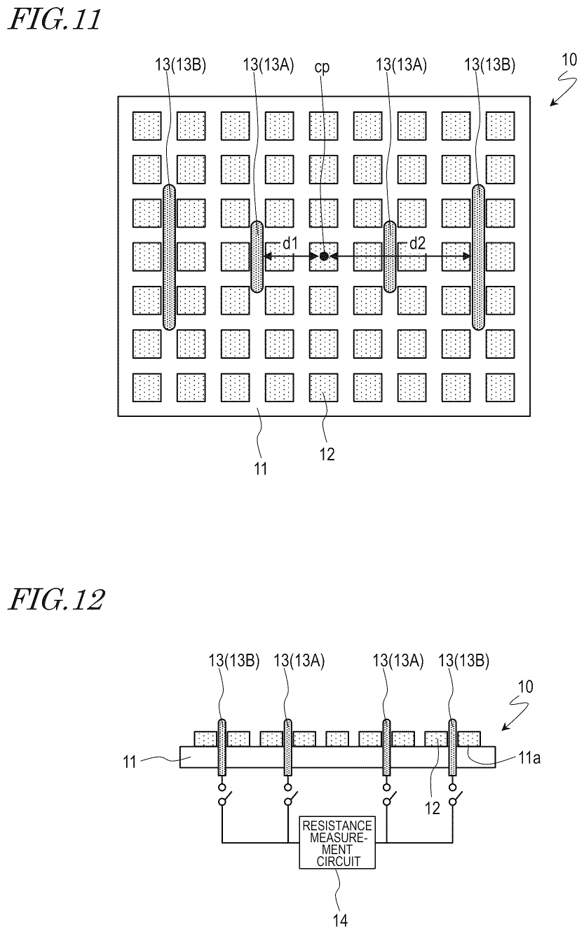

In the example described herein, the plurality of measurement terminals 13 have an equal size (equal area as viewed in plan), although not all the measurement terminals 13 necessarily have an equal size. FIG. 11 and FIG. 12 show another example of the configuration for measuring the flowability of the electric current.

In the example shown in FIG. 11 and FIG. 12, the plurality of measurement terminals 13 include the first terminals 13A and the second terminals 13B, which have different areas as viewed in plan. Herein, the number of first terminals 13A and the number of second terminals 13B are each two.

As viewed in plan, the distance from the center cp of the tactile sensation presenting section 10 to the second terminals 13B, d2, is longer than the distance from the center cp to the first terminals 13A, d1. The area of the second terminals 13B as viewed in plan is greater than the area of the first terminals 13A as viewed in plan.

As the distance between the measurement terminals 13 increases, the electric current path has higher resistance, and there is a probability that it will be necessary to cause a larger electric current to flow. Therefore, it is preferred that, as shown in FIG. 11 and FIG. 12, the area of the second terminals 13B that are relatively distant from the center cp is greater than the area of the first terminals 13A that are relatively close to the center cp. Near the center cp of the tactile sensation presenting section 10, it is highly probable that the center of the fingertip inner portion fp and the tactile sensation presenting section 10 are in tight contact together, while at a position away from the center cp there is a probability that the fingertip inner portion fp will not be in sufficient contact with the tactile sensation presenting section 10 due to the difference in size or roundness of the finger F or the slight deviation of the position at which the finger F is placed. Thus, it is also preferred from this viewpoint that the area of the second terminals 13B is greater than the area of the first terminals 13A.

In the example described herein, the resistance value is measured, although the electric current value may be measured in the presence of a voltage applied between the measurement terminals 13. Alternatively, by measuring the resistance value for each of a plurality of combinations of the measurement terminals 13, the variation of the resistance value due to the difference in interterminal distance may be grasped. The variation of the resistance value due to the difference in applied voltage, the difference in the positional relationship between the measurement terminals 13, or the difference in area of the measurement terminals 13 may be grasped.

The measurement of the flowability of the electric current may be carried out before presentation of tactile sensations, e.g., immediately after the tactile sensation presenting device 100 is powered on or immediately after playback of contents, or may be carried out on a regular basis in the period where tactile sensations are not presented (i.e., in the intervals between the periods where tactile sensations are presented). In the case where tactile sensations are presented in synchronization with the display frames of the video, the flowability of the electric current may always be measured in the frames where tactile sensations are not presented.

In the examples shown in FIG. 9, FIG. 10, FIG. 11 and FIG. 12, the flowability of the electric current is measured using the measurement terminals 13, although the tactile sensation presenting section 10 may not have the measurement terminals 13, and at least two of the plurality of electrodes 12 that are for presentation of tactile sensations may be used to measure the flowability of the electric current.

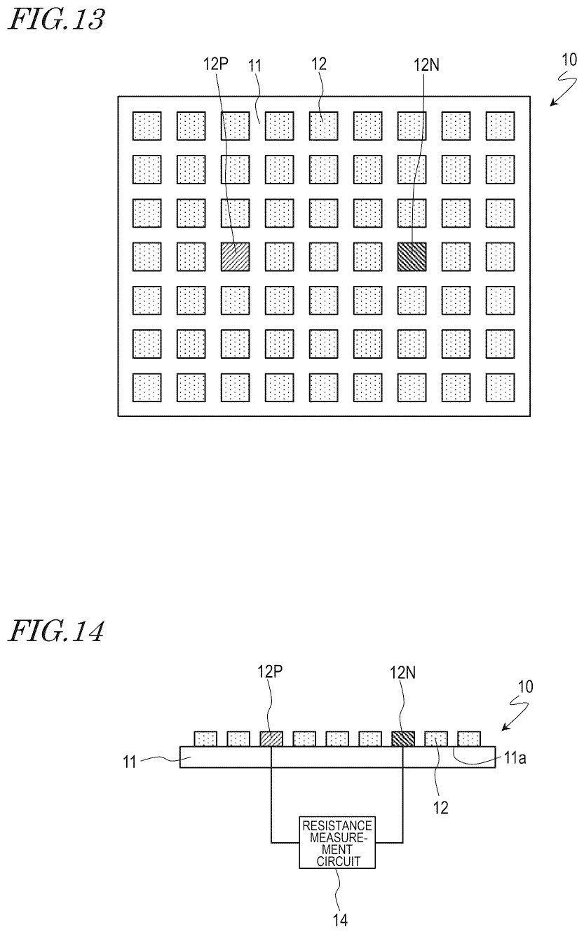

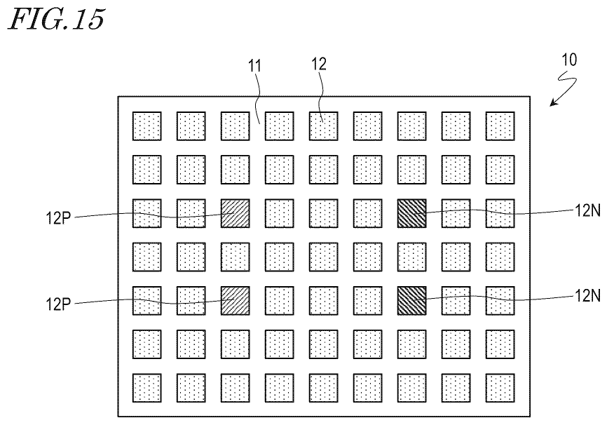

FIG. 13 and FIG. 14 show an example of the configuration for the measurement with the use of the electrodes 12. In the example shown in FIG. 13 and FIG. 14, two (one pair) of the plurality of electrodes 12 are used for the measurement. One predetermined electrode 12P is supplied with a relatively high potential so as to function as a positive terminal. Another one predetermined electrode 12N is supplied with a relatively low potential so as to function as a negative terminal. The electrode 12P that functions as a positive terminal (hereinafter, also referred to as “positive terminal 12P”) and the electrode 12N that functions as a negative terminal (hereinafter, also referred to as “negative terminal 12N”) are electrically coupled with the resistance measurement circuit 14.

In the example shown in FIG. 13 and FIG. 14, the number of positive terminals 12P and the number of negative terminals 12N are each one, although the number of positive terminals 12P and the number of negative terminals 12N may each be two or more. FIG. 15 shows another example of the configuration for the measurement with the use of the electrodes 12.

In the example shown in FIG. 15, the number of positive terminals 12P and the number of negative terminals 12N are each two. Two or more electrodes 12P are used as positive terminals and two or more electrodes 12N are used as negative terminals for the sake of averaging, so that the measurement accuracy can be improved. Even if the contact position of the tactile sensation presenting section 10 and the finger F deviates from the center of the tactile sensation presenting section 10, the measurement can be suitably carried out.

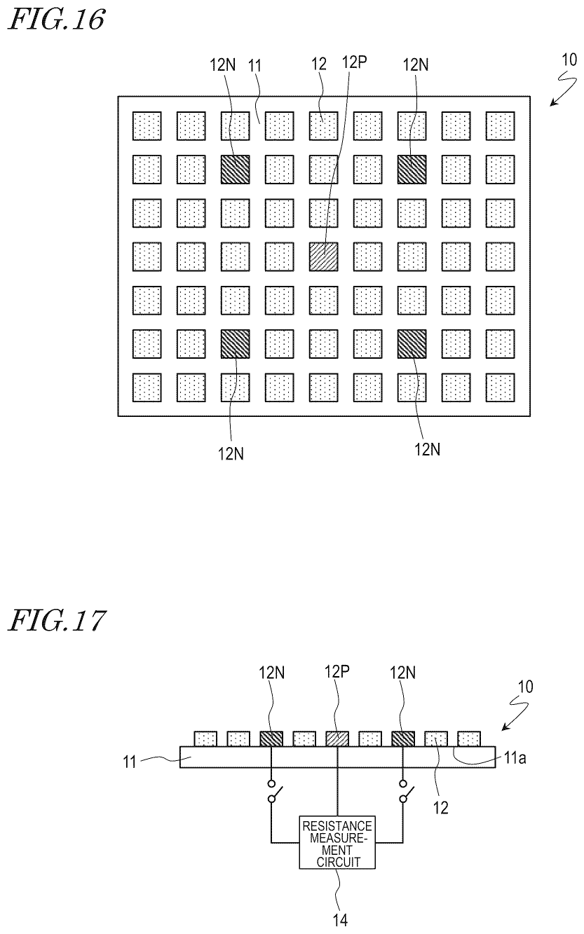

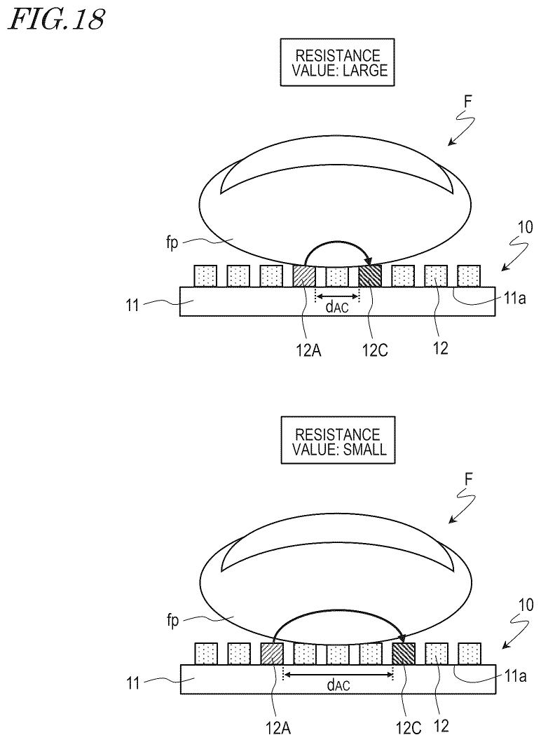

In the examples shown in FIG. 13, FIG. 14 and FIG. 15, the number of positive terminals 12P is equal to the number of negative terminals 12N, although the number of positive terminals 12P and the number of negative terminals 12N may be different from each other. FIG. 16 and FIG. 17 show still another example of the configuration for the measurement with the use of the electrodes 12.

In the example shown in FIG. 16 and FIG. 17, the number of positive terminals 12P is one, and the number of negative terminals 12N is four. The positive terminal 12P is located at the center of the tactile sensation presenting section 10 as viewed in plan, and the negative terminals 12N are located closer to the periphery than the positive terminal 12P. The total of the resistance values between the positive terminal 12P and the four negative terminals 12N may be measured by simultaneously coupling all of the four electrodes 12 that serve as the negative terminals 12N with the resistance measurement circuit 14. Alternatively, the resistance values may be sequentially measured by coupling the electrodes 12 that serve as the negative terminals 12N with the resistance measurement circuit 14 in a one-by-one manner (i.e., sequentially switching the electrodes 12 that serve as the negative terminals 12N).

In the examples shown in FIG. 13, FIG. 14, FIG. 15, FIG. 16 and FIG. 17, few electrodes 12 are used in the measurement, although a larger number of electrodes 12 may be used, or the measurement may be carried out using electrodes 12 at desired positions according to the conditions under which the tactile sensation presenting device 100 is used. Note that, however, in such a case, it is necessary to provide a switch for each of the electrodes 12 for electrical coupling with the resistance measurement circuit 14 and, therefore, the circuit configuration and control can be somewhat complicated.

Note that, also in the case where the measurement of the flowability of the electric current is performed using the electrodes 12, the electric current value may be measured instead of the measurement of the resistance value.

Thus, the measurement of the flowability of the electric current may be performed using the measurement terminals 13 or may be performed using the electrodes 12 that are for presentation of tactile sensations. When the measurement terminals 13 are used as previously described with reference to FIG. 9, FIG. 10, FIG. 11 and FIG. 12, high accuracy measurement with low noise can be easily realized, and the measurement can be easily performed without the necessity for complicated control. In comparison, when the electrodes 12 for presentation of tactile sensations are used, the measurement position can be changed relatively flexibly, and adequate measurement can be performed according to the contact state of the finger F.

### [Adjustment of Execution Mode of Electrical Stimulation]

The control section 20 adjusts the execution mode of electrical stimulation (hereinafter, also referred to as “adjustment of stimulation mode”) by, for example, performing at least one of the following adjustments (1), (2) and (3). Thereby, optimum presentation of tactile sensations can be achieved.
(1) Adjustment of the distance between the anode 12A and the cathode 12C.
(2) Adjustment of the total area of the electrodes 12 which function as the anodes 12A and the total area of the electrodes 12 which function as the cathodes 12C.
(3) Adjustment of the pattern of the voltage applied to the electrodes 12 which function as the anodes 12A and the electrodes 12 which function as the cathodes 12C.

Hereinafter, adjustments (1), (2) and (3) are described in order.

### [Adjustment (1)]

First, the adjustment of the distance between the anode 12A and the cathode 12C (hereinafter, referred to as “anode-cathode distance”), d_{AC}, is described with reference to FIG. 18. FIG. 18 illustrates a setting example of the anode 12A and the cathode 12C. The upper part of FIG. 18 shows a case where the measured resistance value is relatively large (i.e., the flowability of the electric current is relatively low). The lower part of FIG. 18 shows a case where the measured resistance value is relatively small (i.e., the flowability of the electric current is relatively high).

In the example shown in the upper part of FIG. 18, the fourth and sixth electrodes 12 from the left end of the shown row are set as the anode 12A and the cathode 12C, respectively. In the example shown in the lower part of FIG. 18, the third and seventh electrodes 12 from the left end of the shown row are set as the anode 12A and the cathode 12C, respectively. Therefore, the anode-cathode distance d_{AC} is relatively short when the resistance value is relatively large, but the anode-cathode distance d_{AC} is relatively long when the resistance value is relatively small.

It can be said that the flowability of the electric current increases as the anode-cathode distance d_{AC} decreases, but the flowability of the electric current decreases as the anode-cathode distance d_{AC} increases. Therefore, when the resistance value is relatively large, the flowability of the electric current can be increased by decreasing the anode-cathode distance d_{AC}. On the other hand, when the resistance value is relatively small, the anode-cathode distance d_{AC} may be increased. In either case, if an electric current at a predetermined value or higher is allowed to flow, electrical stimulation can be perceived as a tactile sensation. Note that, if the tactile sensation varies depending on the difference in the anode-cathode distance d_{AC} even with the same electric current value, the target electric current value may be adjusted according to the anode-cathode distance d_{AC}.

Adjustment of the anode-cathode distance d_{AC} is performed based on the measured flowability of the electric current. If the measured resistance value is excessively small, an excessive electric current flows and there is a probability that it will cause safety concerns. If the measured resistance value is excessively large, there is a probability that no electric current will flow. Within such a set voltage range that the electric current flows stably to some extent, if the measured resistance value is large, the anode-cathode distance d_{AC} may be decreased; but if the measured resistance value is small, the anode-cathode distance d_{AC} may be increased.

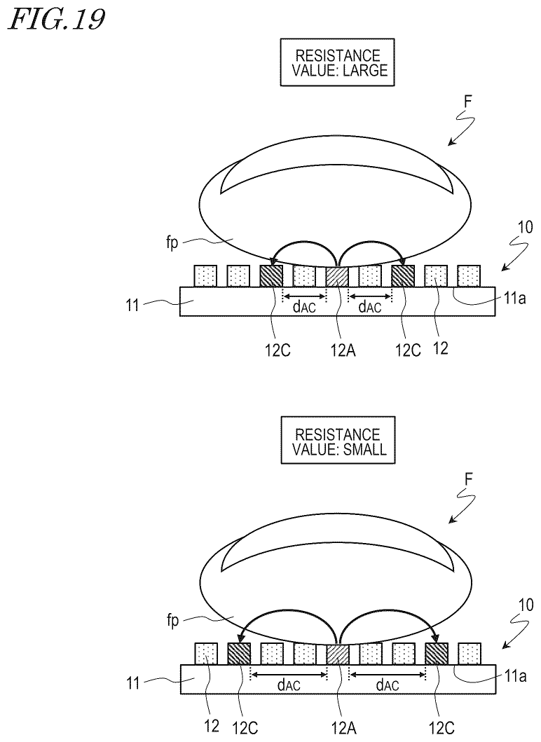

Note that a plurality of cathodes 12C may be set for a single anode 12A. FIG. 19 illustrates another setting example of the anode 12A and the cathodes 12C. The upper part of FIG. 19 shows a case where the measured resistance value is relatively large. The lower part of FIG. 19 shows a case where the measured resistance value is relatively small.

In the example shown in the upper part of FIG. 19, the fifth electrode 12 from the left end of the shown row is set as the anode 12A, and the third and seventh electrodes 12 are set as the cathodes 12C. On the other hand, in the example shown in the lower part of FIG. 19, the fifth electrode 12 from the left end of the shown row is set as the anode 12A, and the second and eighth electrodes 12 are set as the cathodes 12C. Therefore, the anode-cathode distance d_{AC} is relatively short when the resistance value is relatively large, but the anode-cathode distance d_{AC} is relatively long when the resistance value is relatively small.

Thus, even when a plurality of cathodes 12C are set for a single anode 12A, the anode-cathode distance d_{AC} may be adjusted according to the measured resistance value.

In the examples shown in FIG. 18 and FIG. 19, for simplicity of description, nine electrodes 12 are arrayed in a row in the transverse direction in the drawings, although providing a larger number of electrodes 12 is preferred from the viewpoint of adjusting the anode-cathode distance d_{AC} with higher resolution. For example, in a single tactile sensation presenting section 10, each row and each column may include 64 electrodes 12. In this case, if the width (dimension) along the row direction and the width (dimension) along the column direction of each electrode 12 are 0.12 mm and the array pitch along the row direction and the array pitch along the column direction of the electrodes 12 are 0.16 mm (i.e., the gap between the electrodes 12 is 0.04 mm), the transverse width (transverse dimension) and the longitudinal width (longitudinal dimension) of the tactile sensation presenting section 10 are about 10.2 mm. As a matter of course, the dimensions are not limited to this example. For example, in a single tactile sensation presenting section 10, each row and each column may include 16 electrodes 12. In this case, if the width (dimension) along the row direction and the width (dimension) along the column direction of each electrode 12 are 0.30 mm and the array pitch along the row direction and the array pitch along the column direction of the electrodes 12 are 0.66 mm (i.e., the gap between the electrodes 12 is 0.36 mm), the transverse width (transverse dimension) and the longitudinal width (longitudinal dimension) of the tactile sensation presenting section 10 are about 10.2 mm. Alternatively, a TFT array substrate, which is capable of controlling the voltage or electric current with such a pixel density as those of liquid crystal display devices and organic EL (OLED) display devices, may be used as the tactile sensation presenting section 10.

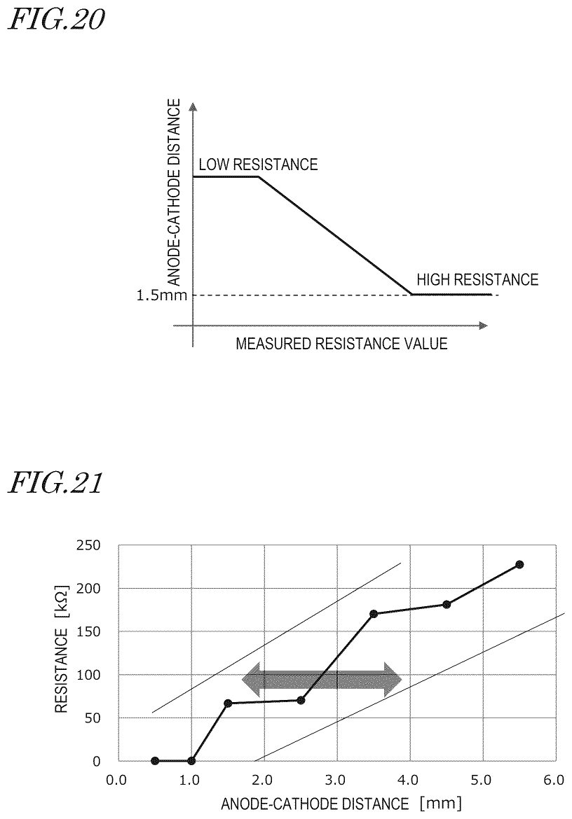

FIG. 20 shows an example of the relationship between the resistance value and the anode-cathode distance d_{AC} in a case where the anode-cathode distance d_{AC} is adjusted according to the measured resistance value. The control section 20 controls the tactile sensation presenting section 10 such that the anode-cathode distance d_{AC} is increased as the measured resistance value decreases and that the anode-cathode distance d_{AC} is decreased as the measured resistance value increases, such as in the example shown in FIG. 20, whereby the electric current flowing through the fingertip inner portion fp can be made constant irrespective of the individual difference in internal resistance or the environment in which the device is used. For example, the tactile sensation presenting device 100 may have a table indicative of the relationship between the flowability (resistance value) of the electric current and the anode-cathode distance d_{AC} such as shown in FIG. 20, which is stored in advance. After the flowability of the electric current is measured, the control section 20 may acquire the anode-cathode distance d_{AC} corresponding to the measured resistance value based on the above-described table and set the electrodes 12 which function as the anodes 12A and the electrodes 12 which function as the cathodes 12C such that the acquired anode-cathode distance d_{AC} is achieved. Note that, such as in the example shown in FIG. 20, the upper limit value and the lower limit value may be placed on the anode-cathode distance d_{AC}. Alternatively, the anode-cathode distance d_{AC} may be changed stepwise according to the flowability of the electric current. Different tables may be used for different users.

TABLE 1 shows the results of measurements of the electric current actually flowing through the finger F of subjects and the tactile sensation value with varying anode-cathode distances d_{AC}. Herein, the tactile sensation value is a sensation value obtained by subjective evaluation by human test subjects, specifically, a value recorded based on subjective evaluation of the degree of physical projection sensed when electrical stimulation was applied to fingertips.

TABLE 1

| Set values | | | Measured values | | |
|---|---|---|---|---|---|
| Anode- | | | | | Tactile |
| cathode | | | Electric | | sensation |
| distance | Frequency | Voltage | current | Resistance | value |
| (mm) | (Hz) | (Vrms) | (μA) | (kΩ) | (mm) |
| 5.5 | 200 | 40 | 176 | 227.3 | 1.5 |
| 4.5 | | | 221 | 181.0 | 1.5 |
| 3.5 | | | 235 | 170.2 | 2.0 |
| 2.5 | | | 570 | 70.2 | 2.5 |
| 1.5 | | | 600 | 66.7 | 2.0 |
| 1.0 | | | 0 | — | 0.0 |
| 0.5 | | | 0 | — | 0.0 |

As seen from TABLE 1, as the anode-cathode distance d_{AC} decreases, the electric current value increases, and the resistance value decreases. The tactile sensation value was largest when the anode-cathode distance d_{AC} was 2.5 mm.

The relationship between the anode-cathode distance d_{AC} and the resistance value in TABLE 1 is shown in FIG. 21. It can be said that the individual difference in the flowability of the electric current through the human finger F is the variation as indicated by the double headed arrow in FIG. 21. Thus, the anode-cathode distance d_{AC} may be adjusted according to the measured resistance value such that an electric current can flow which has a desired magnitude corresponding to a tactile sensation intended to be presented.

As also seen from the measurement results of the tactile sensation value shown in TABLE 1, the tactile sensation is perceived most intensely when the anode-cathode distance d_{AC} is 2.5 mm, rather than such a simple relationship that the tactile sensation is perceived more intensely as the anode-cathode distance d_{AC} decreases or as the electric current value increases. Thus, it is preferred that, in addition to setting the electric current value to a desired value, the anode-cathode distance d_{AC} is set in consideration of the intensity of perception of tactile sensations which depends on the anode-cathode distance d_{AC} (for example, a table indicative of the relationship between the resistance value and the anode-cathode distance d_{AC} is prepared).

As described above, the control section 20 measures the flowability of the electric current and adjusts the anode-cathode distance d_{AC} based on the results of the measurement, so that optimum and safe supply of the voltage/current can be achieved irrespective of the individual difference in internal resistance or the environment in which the device is used, and optimum electrical stimulation can be provided to sensory receptors under the skin.

When a plurality of electrodes 12 arrayed in a matrix (matrix electrodes) such as those described above are used, the positional relationship between the anode 12A and the cathode(s) 12C and the anode-cathode distance d_{AC} can be changed, so that better settings can be achieved. Further, when high-resolution (high-density) matrix electrodes are used, the resolution of the set distance can be increased, and presentation of tactile sensations with higher resolutions can be achieved.

### [Adjustment (2)]

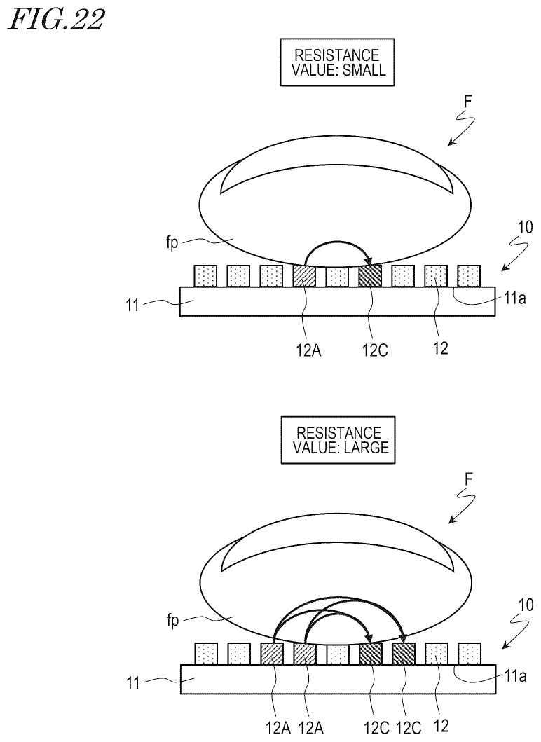

Next, adjustment of the total area of electrodes 12 which function as the anodes 12A (hereinafter, also simply referred to as “the total area of the anodes 12A”) and the total area of electrodes 12 which function as the cathodes 12C (hereinafter, also simply referred to as “the total area of the cathodes 12C”) is described with reference to FIG. 22. FIG. 22 illustrates a setting example of the anodes 12A and the cathodes 12C. The upper part of FIG. 22 shows a case where the measured resistance value is relatively small (i.e., the flowability of the electric current is relatively high). The lower part of FIG. 22 shows a case where the measured resistance value is relatively large (i.e., the flowability of the electric current is relatively low).

In the example shown in the upper part of FIG. 22, the fourth and sixth electrodes 12 from the left end of the shown row are set as the anode 12A and the cathode 12C, respectively. That is, a pair of electrodes 12 are used as the anode 12A and the cathode 12C. On the other hand, in the example shown in the lower part of FIG. 22, the third and fourth electrodes 12 from the left end of the shown row are set as the anodes 12A, and the sixth and seventh electrodes 12 are set as the cathodes 12C. That is, two pairs of electrodes 12 are used as the anodes 12A and the cathodes 12C. Therefore, the number of electrodes 12 which function as the anodes 12A (hereinafter, also simply referred to as “the number of anodes 12A”) and the number of electrodes 12 which function as the cathodes 12C (hereinafter, also simply referred to as “the number of cathodes 12C”) are relatively small when the resistance value is relatively small, but relatively large when the resistance value is relatively large. In other words, the total area of the anodes 12A and the total area of the cathodes 12C are relatively small when the resistance value is relatively small, but relatively large when the resistance value is relatively large.

As the total area of the anodes 12A and the total area of the cathodes 12C increase, the magnitude of the electric current can be increased. Therefore, when the resistance value is relatively large, an electric current of a sufficient magnitude can be secured by increasing the total area of the anodes 12A and the total area of the cathodes 12C. On the other hand, when the resistance value is relatively small, the total area of the anodes 12A and the total area of the cathodes 12C may be decreased.

The adjustment of the total area of the anodes 12A and the total area of the cathodes 12C (the adjustment of the number of anodes 12A and the number of cathodes 12C) is performed based on the measured flowability of the electric current. If the measured resistance value is excessively small, an excessive electric current flows and there is a probability that it will cause safety concerns. If the measured resistance value is excessively large, there is a probability that no electric current will flow. Within such a set voltage range that the electric current flows stably to some extent, if the measured resistance value is large, the total area of the anodes 12A and the total area of the cathodes 12C may be increased (i.e., the number of anodes 12A and the number of cathodes 12C may be increased); but if the measured resistance value is small, the total area of the anodes 12A and the total area of the cathodes 12C may be decreased (i.e., the number of anodes 12A and the number of cathodes 12C may be decreased).

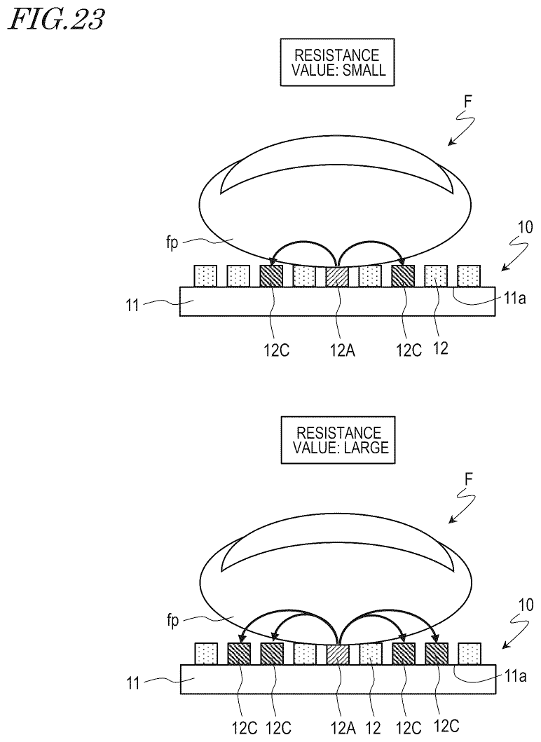

Note that a plurality of cathodes 12C may be set for a single anode 12A. FIG. 23 illustrates another setting example of the anode 12A and the cathodes 12C. The upper part of FIG. 23 shows a case where the measured resistance value is relatively small. The lower part of FIG. 23 shows a case where the measured resistance value is relatively large.

In the example shown in the upper part of FIG. 23, the fifth electrode 12 from the left end of the shown row is set as the anode 12A, and the third and seventh electrodes 12 are set as the cathodes 12C. On the other hand, in the example shown in the lower part of FIG. 23, the fifth electrode 12 from the left end of the shown row is set as the anode 12A, and the second, third, seventh and eighth electrodes 12 are set as the cathodes 12C. In this case, it can be said that the total area of the anodes 12A and the total area of the cathodes 12C are adjusted, and it can also be said that the ratio between the total area of the anodes 12A and the total area of the cathodes 12C is adjusted (the ratio between the number of anodes 12A and the number of cathodes 12C is adjusted).

In the examples shown in FIG. 22 and FIG. 23, for simplicity of description, nine electrodes 12 are arrayed in a row in the transverse direction in the drawings, although providing a larger number of electrodes 12 is preferred from the viewpoint of adjusting the total area of the anodes 12A and the total area of the cathodes 12C (the number of anodes 12A and the number of cathodes 12C) with higher resolution. For example, in a single tactile sensation presenting section 10, each row and each column may include 64 electrodes 12. In this case, if the width (dimension) along the row direction and the width (dimension) along the column direction of each electrode 12 are 0.12 mm and the array pitch along the row direction and the array pitch along the column direction of the electrodes 12 are 0.16 mm (i.e., the gap between the electrodes 12 is 0.04 mm), the transverse width (transverse dimension) and the longitudinal width (longitudinal dimension) of the tactile sensation presenting section 10 are about 10.2 mm. As a matter of course, the dimensions are not limited to this example. For example, in a single tactile sensation presenting section 10, each row and each column may include 16 electrodes 12. In this case, if the width (dimension) along the row direction and the width (dimension) along the column direction of each electrode 12 are 0.30 mm and the array pitch along the row direction and the array pitch along the column direction of the electrodes 12 are 0.66 mm (i.e., the gap between the electrodes 12 is 0.36 mm), the transverse width (transverse dimension) and the longitudinal width (longitudinal dimension) of the tactile sensation presenting section 10 are about 10.2 mm. Alternatively, a TFT array substrate, which is capable of controlling the voltage or electric current with such a pixel density as those of liquid crystal display devices and organic EL (OLED) display devices, may be used as the tactile sensation presenting section 10.

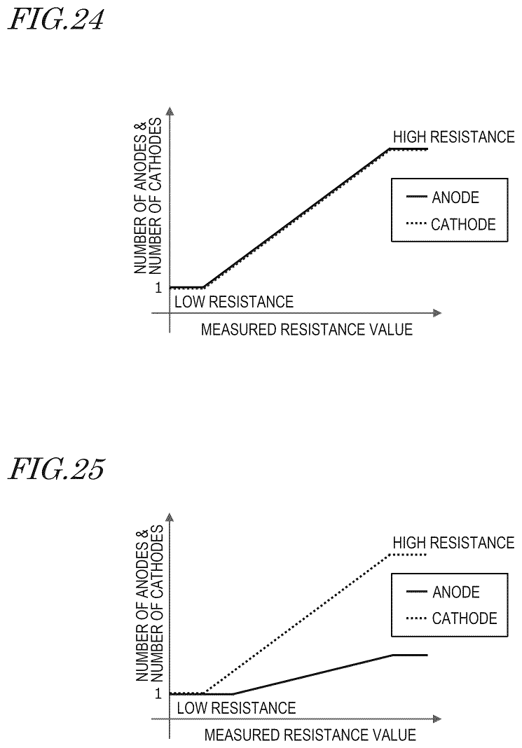

FIG. 24 shows an example of the relationship between the resistance value and the number of anodes 12A and the number of cathodes 12C in a case where the number of anodes 12A and the number of cathodes 12C are adjusted according to the measured resistance value. In the example shown in FIG. 24, the number of anodes 12A is equal to the number of cathodes 12C. The control section 20 controls the tactile sensation presenting section 10 such that the number of anodes 12A and the number of cathodes 12C are increased as the measured resistance value increases and that the number of anodes 12A and the number of cathodes 12C are decreased as the measured resistance value decreases, such as in the example shown in FIG. 24, whereby the electric current flowing through the fingertip inner portion fp can be made constant irrespective of the individual difference in internal resistance or the environment in which the device is used.

FIG. 25 shows an example of the relationship between the resistance value and the number of anodes 12A and the number of cathodes 12C in a case where the ratio between the number of anodes 12A and the number of cathodes 12C is adjusted according to the measured resistance value. The control section 20 controls the tactile sensation presenting section 10 such that the number of anodes 12A and the number of cathodes 12C are increased as the measured resistance value increases and that the number of cathodes 12C is larger than the number of anodes 12A, such as in the example shown in FIG. 25. Contrary to the example shown in FIG. 25, the tactile sensation presenting section 10 may be controlled such that the number of anodes 12A is larger than the number of cathodes 12C or may be controlled such that one of the number of anodes 12A and the number of cathodes 12C is kept unchanged.

Similarly to the case of Adjustment (1), for example, the tactile sensation presenting device 100 may have a table indicative of the relationship between the flowability (resistance value) of the electric current and the number of anodes 12A and the number of cathodes 12C such as shown in FIG. 24 or FIG. 25, which is stored in advance. After the flowability of the electric current is measured, the control section 20 may acquire the number of anodes 12A and the number of cathodes 12C corresponding to the measured resistance value based on the above-described table and set the electrodes 12 which function as the anodes 12A and the electrodes 12 which function as the cathodes 12C such that the acquired number of anodes 12A and the acquired number of cathodes 12C are achieved. Such as in the example shown in FIG. 24 or FIG. 25, the upper limit value and the lower limit value may be placed on the number of anodes 12A and the number of cathodes 12C (in this case, the lower limit value may be two or more). Alternatively, the number of anodes 12A and the number of cathodes 12C may be changed stepwise according to the flowability of the electric current. Different tables may be used for different users.

As described above, the control section 20 measures the flowability of the electric current and adjusts the total area of the anodes 12A and the total area of the cathodes 12C (adjusts the number of anodes 12A and the number of cathodes 12C) based on the results of the measurement, so that optimum and safe supply of the voltage/current can be achieved irrespective of the individual difference in internal resistance or the environment in which the device is used, and optimum electrical stimulation can be provided to sensory receptors under the skin.

The number of anodes 12A and the number of cathodes 12C may be equal to each other or may be different from each other. We actually conducted verification and found that, when the number of cathodes 12C is larger than the number of anodes 12A, electrical stimulation is more likely to be perceived as a tactile sensation.

When matrix electrodes such as those described above are used, the positional relationship between the anode(s) 12A and the cathode (s) 12C, the shape of the group of electrodes 12 which function as the anode(s) 12A and the cathode(s) 12C, etc., can be flexibly changed, so that better settings can be achieved. Further, when high-resolution (high-density) matrix electrodes are used, the resolution of the set area can be increased, and presentation of tactile sensations with higher resolutions can be achieved.

### [Adjustment (3)]

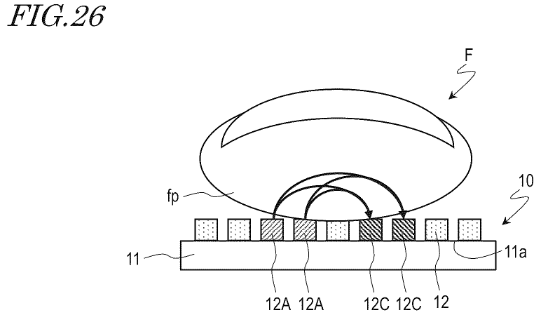

Next, adjustment of the pattern of the voltage applied to the electrodes 12 which function as the anodes 12A and the electrodes 12 which function as the cathodes 12C is described. In the example described hereinafter, the anodes 12A and the cathodes 12C are set as shown in FIG. 26. In the example shown in FIG. 26, the third and fourth electrodes 12 from the left end of the shown row are set as the anodes 12A, and the sixth and seventh electrodes 12 are set as the cathodes 12C. In the following description, the setting of the anodes 12A and the cathodes 12C is not changed according to the measured resistance value although, as a matter of course, it may be changed according to the measured resistance value (i.e., Adjustment (3) may be performed in combination with Adjustment (1) and/or Adjustment (2)).

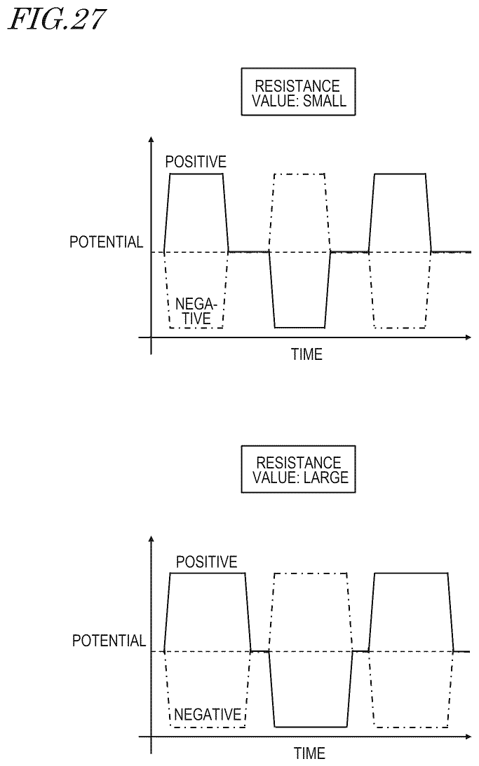

FIG. 27 shows examples of the voltage pattern. The upper part of FIG. 27 shows a case where the measured resistance value is relatively small (i.e., the flowability of the electric current is relatively high). The lower part of FIG. 27 shows a case where the measured resistance value is relatively large (i.e., the flowability of the electric current is relatively low).

As seen from the comparison between the example shown in the upper part of FIG. 27 and the example shown in the lower part of FIG. 27, the duration of application of the voltage to the electrodes 12 which function as the anodes 12A and the electrodes 12 which function as the cathodes 12C is relatively short when the resistance value is relatively small, but relatively long when the resistance value is relatively large. The magnitude of the electric current can be increased as the duration of application of the voltage increases and, in addition, the electric current is more likely to reach a deeper portion under the skin as the duration of application of the voltage increases, so that tactile sensations are more likely to be perceived.

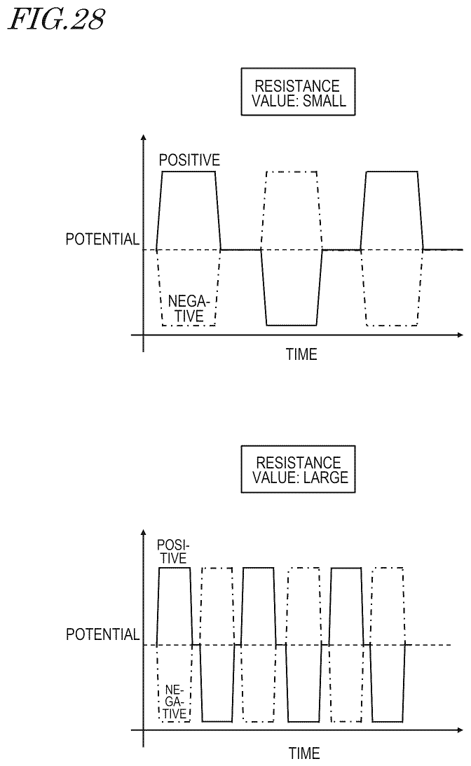

FIG. 28 shows other examples of the voltage pattern. The upper part of FIG. 28 shows a case where the measured resistance value is relatively small. The lower part of FIG. 28 shows a case where the measured resistance value is relatively large.

As seen from the comparison between the example shown in the upper part of FIG. 28 and the example shown in the lower part of FIG. 28, the period of reversal of the polarity (positive/negative) of the potential is relatively long when the resistance value is relatively small, but relatively short when the resistance value is relatively large. The number of reversals of the polarity (positive/negative) of the potential is relatively small when the resistance value is relatively small, but relatively large when the resistance value is relatively large. In this way, in the example shown in FIG. 28, the frequency of the voltage applied to the electrodes 12 which function as the anodes 12A and the electrodes 12 which function as the cathodes 12C is adjusted. By increasing the frequency of the applied voltage, the electric current is more likely to reach a deeper portion under the skin and is more likely to be perceived as a tactile sensation.

FIG. 27 and FIG. 28 show rectangular wave patterns, although the voltage pattern is not limited to rectangular wave patterns but may be sine wave patterns, for example.

If the frequency of the applied voltage changes, there is a probability that the perceived tactile sensation will change. Also, if the frequency does not reach somewhat high levels, there is a probability that some users will not perceive electrical stimulation as a tactile sensation. Therefore, based on the data collected from various types of people in advance, the duration of application and the frequency of the voltage may be adjusted in combination.

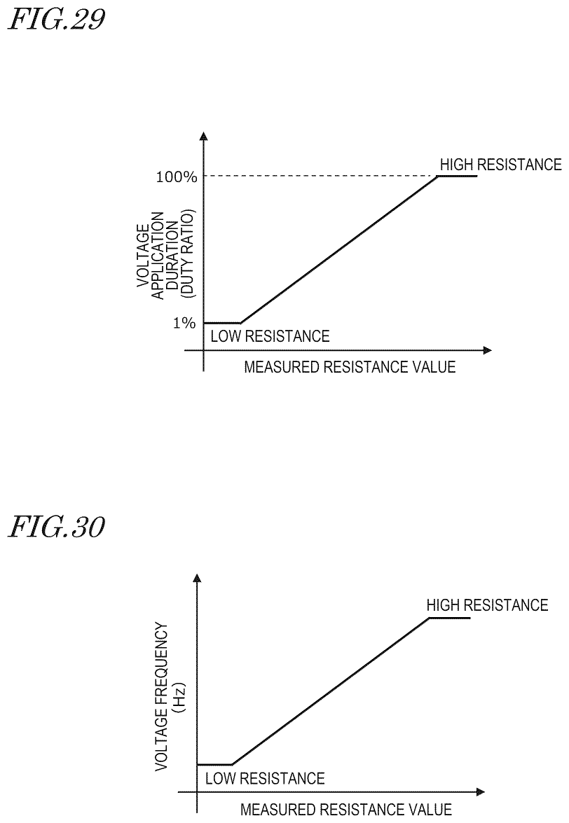

FIG. 29 shows an example of the relationship between the resistance value and the application duration (duty ratio) of the voltage in a case where the application duration of the voltage is adjusted according to the measured resistance value. The control section 20 controls the tactile sensation presenting section 10 such that the application duration of the voltage is increased as the measured resistance value increases and that the application duration of the voltage is decreased as the measured resistance value decreases, such as in the example shown in FIG. 29, whereby the electric current flowing through the fingertip inner portion fp can be made constant irrespective of the individual difference in internal resistance or the environment in which the device is used.

FIG. 30 shows an example of the relationship between the resistance value and the frequency (Hz) of the voltage in a case where the frequency of the voltage is adjusted according to the measured resistance value. The control section 20 controls the tactile sensation presenting section 10 such that the frequency of the voltage is increased as the measured resistance value increases and that the frequency of the voltage is decreased as the measured resistance value decreases, such as in the example shown in FIG. 30, whereby the electric current flowing through the fingertip inner portion fp can be made constant irrespective of the individual difference in internal resistance or the environment in which the device is used. Note that both the application duration and frequency of the voltage may be adjusted.

Similarly to the case of Adjustment (1), for example, the tactile sensation presenting device 100 may have a table indicative of the relationship between the flowability (resistance value) of the electric current and the application pattern of the voltage (the application duration of the voltage and/or the frequency of the voltage) such as shown in FIG. 29 and/or FIG. 30, which is stored in advance. After the flowability of the electric current is measured, the control section 20 may acquire the application pattern of the voltage corresponding to the measured resistance value based on the above-described table and apply a voltage of the acquired application pattern to the anodes 12A and/or the cathodes 12C. Such as in the example shown in FIG. 30, the upper limit value and the lower limit value may be placed on the frequency of the voltage. Alternatively, the application pattern of the voltage may be changed stepwise according to the flowability of the electric current. Different tables may be used for different users.

As described above, the control section 20 measures the flowability of the electric current and adjusts the pattern of the voltage applied to the electrodes 12 which function as the anodes 12A and the electrodes 12 which function as the cathodes 12C based on the results of the measurement, so that optimum and safe supply of the voltage/current can be achieved irrespective of the individual difference in internal resistance or the environment in which the device is used, and optimum electrical stimulation can be provided to sensory receptors under the skin.

When adjustment of the voltage pattern (Adjustment (3)) is performed in combination with Adjustment (1) (adjustment of the anode-cathode distance d_{AC}) and/or Adjustment (2) (adjustment of the total area of the anodes 12A and the total area of the cathodes 12C) which have been previously described, the adjustment can be performed with higher flexibility, so that better presentation of tactile sensations can be achieved.

### [Other Electrode Structures]

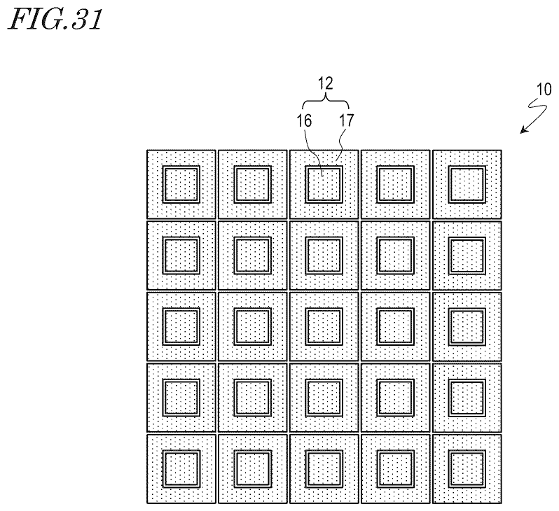

Another example of the electrode structure of the tactile sensation presenting section 10 is described with reference to FIG. 31. In the example shown in FIG. 31, the plurality of electrodes 12 of the tactile sensation presenting section 10 include a plurality of electrodes for anode (hereinafter, referred to as “anodic electrodes 16”) and a plurality of electrodes for cathode (hereinafter, referred to as “cathodic electrodes 17”).

The plurality of anodic electrodes 16 are provided on the major surface of the support (not shown herein). The plurality of anodic electrodes 16 are arrayed in a matrix.

The plurality of cathodic electrodes 17 are provided on the major surface of the support so as not to overlap the plurality of anodic electrodes 16 as viewed in plan. The plurality of cathodic electrodes 17 are also arrayed in a matrix. In the illustrated example, each of the cathodic electrodes 17 has such a shape that surrounds a single anodic electrode 16 as viewed in plan. Note that, although not shown herein, each of the cathodic electrodes 17 may have such a shape that surrounds two or more anodic electrodes 16.

The control section 20 is capable of independently switching each of the plurality of anodic electrodes 16 between a state where the anodic electrode 16 is supplied with the anodic potential and a state where the anodic electrode 16 is supplied with a floating potential. Also, the control section 20 is capable of independently switching each of the plurality of cathodic electrodes 17 between a state where the cathodic electrode 17 is supplied with the cathodic potential and a state where the cathodic electrode 17 is supplied with a floating potential.

As described above, in the example shown in FIG. 31, electrodes which function as the anodes 12A (anodic electrodes 16) and electrodes which function as the cathodes 12C (cathodic electrodes 17) are separately provided. Since the anodic electrodes 16 and the cathodic electrodes 17 are separately provided, wire structures can be easily formed, and application of the voltage to each of the electrodes 12 can be easily controlled.

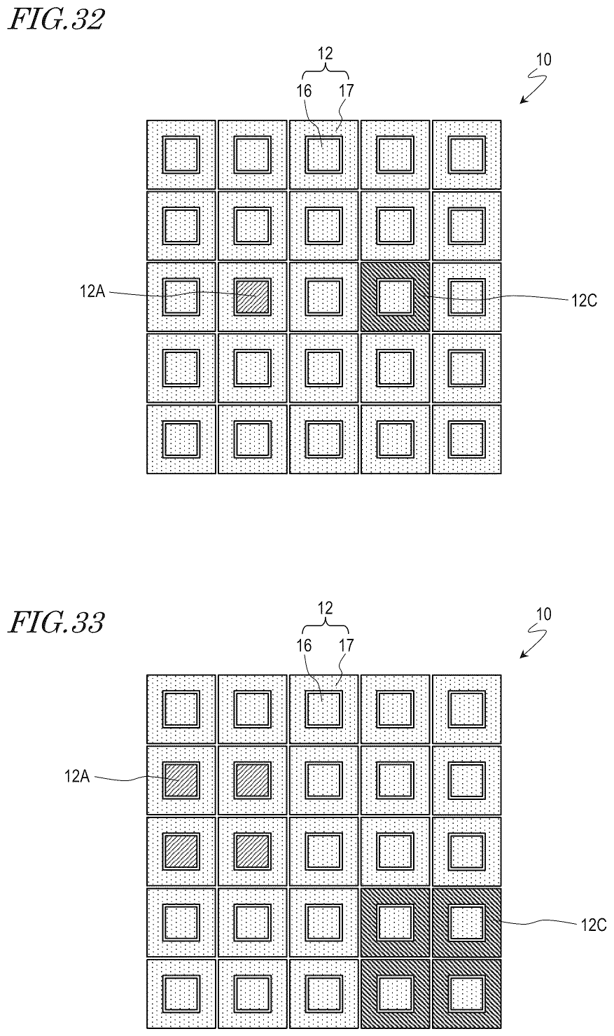

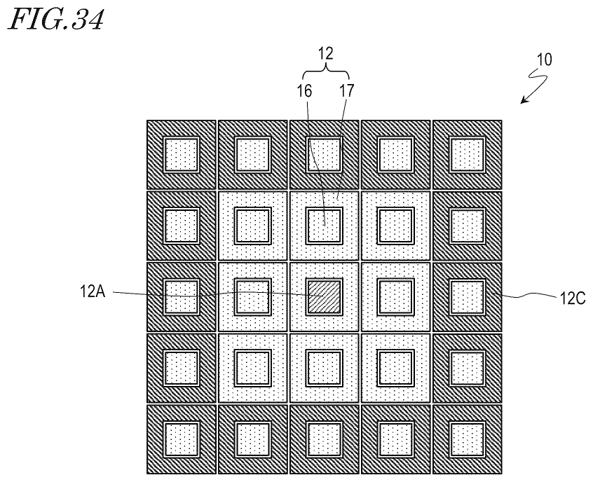

FIG. 32, FIG. 33 and FIG. 34 show setting examples of the anode(s) 12A and the cathode(s) 12C in the case where the electrode structure shown in FIG. 31 is employed.

In the example shown in FIG. 32, the anodic electrode 16 located at the intersection of the third row and the second column is supplied with the anodic potential and functions as the anode 12A, while the cathodic electrode 17 located at the intersection of the third row and the fourth column is supplied with the cathodic potential and functions as the cathode 12C. The other anodic electrodes 16 and the other cathodic electrodes 17 are supplied with a floating potential.

In the example shown in FIG. 33, the anodic electrodes 16 located at the intersection of the second row and the first column, the intersection of the second row and the second column, the intersection of the third row and the first column, and the intersection of the third row and the second column are supplied with the anodic potential and function as the anodes 12A, while the cathodic electrodes 17 located at the intersection of the fourth row and the fourth column, the intersection of the fourth row and the fifth column, the intersection of the fifth row and the fourth column, and the intersection of the fifth row and the fifth column are supplied with the cathodic potential and function as the cathodes 12C. The other anodic electrodes 16 and the other cathodic electrodes 17 are supplied with a floating potential.

In the example shown in FIG. 34, the anodic electrode 16 located at the intersection of the third row and the third column is supplied with the anodic potential and function as the anodes 12A, while the cathodic electrodes 17 located in the first row, the fifth row, the first column and the fifth column are supplied with the cathodic potential and function as the cathodes 12C. The other anodic electrodes 16 and the other cathodic electrodes 17 are supplied with a floating potential.

As also seen from FIG. 32, FIG. 33 and FIG. 34, even in the case where the electrode structure shown in FIG. 31 is employed, adjustment of the anode-cathode distance d_{AC}, adjustment of the total area of the anodes 12A and the total area of the cathodes 12C (in other words, adjustment of the number of anodes 12A and the number of cathodes 12C), adjustment of the ratio between the total area of the anodes 12A and the total area of the cathodes 12C (in other words, adjustment of the ratio between the number of anodes 12A and the number of cathodes 12C), and adjustment of the pattern of the voltage applied to the electrodes 12 which function as the anodes 12A and the electrodes 12 which function as the cathodes 12C can be flexibly performed.

Embodiments of the present invention are widely applicable to electrical stimulation type tactile sensation presenting devices that are configured to present tactile sensations by electrical stimulation.

This application is based on Japanese Patent Application No. 2023-185367 filed on Oct. 30, 2023, the entire contents of which are hereby incorporated by reference.

What is claimed is:

1. A tactile sensation presenting device comprising:
a tactile sensation presenting section having a plurality of electrodes to present a tactile sensation by electrical stimulation at a specific portion of a user; and
a control section capable of controlling the tactile sensation presenting section to provide electrical stimulation using at least one of the plurality of electrodes as an anode and at least another one of the plurality of electrodes as a cathode,
wherein the control section measures a flowability of an electric current through the portion and adjusts an execution mode of the electrical stimulation based on a result of the measurement.

2. The tactile sensation presenting device of claim 1, wherein
the tactile sensation presenting section further includes a plurality of measurement terminals, and
the control section performs the measurement using the plurality of measurement terminals.

3. The tactile sensation presenting device of claim 2, wherein the plurality of measurement terminals include a first terminal and a second terminal, the first terminal and the second terminal having different areas as viewed in plan.

4. The tactile sensation presenting device of claim 3, wherein
a distance from a center of the tactile sensation presenting section to the second terminal is greater than a distance from the center to the first terminal as viewed in plan, and
the area of the second terminal as viewed in plan is greater than the area of the first terminal as viewed in plan.

5. The tactile sensation presenting device of claim 1, wherein the control section performs the measurement using at least two of the plurality of electrodes.

6. The tactile sensation presenting device of claim 5, wherein the number of electrodes which function as positive terminals in the measurement and the number of electrodes which function as negative terminals in the measurement are each two or more.

7. The tactile sensation presenting device of claim 5, wherein the number of electrodes which function as positive terminals in the measurement and the number of electrodes which function as negative terminals in the measurement are different from each other.

8. The tactile sensation presenting device of claim 1 wherein, in adjusting the execution mode of the electrical stimulation, the control section adjusts a distance between the anode and the cathode.

9. The tactile sensation presenting device of claim 1 wherein, in adjusting the execution mode of the electrical stimulation, the control section adjusts a total area of the electrode which functions as the anode and a total area of the electrode which functions as the cathode.

10. The tactile sensation presenting device of claim 9 wherein, in adjusting the execution mode of the electrical stimulation, the control section adjusts a ratio between the total area of the electrode which functions as the anode and the total area of the electrode which functions as the cathode.

11. The tactile sensation presenting device of claim 1 wherein, in adjusting the execution mode of the electrical stimulation, the control section adjusts a pattern of a voltage applied to the electrode which functions as the anode and the electrode which functions as the cathode.

12. The tactile sensation presenting device of claim 11 wherein, in adjusting the execution mode of the electrical stimulation, the control section adjusts an application duration of the voltage applied to the electrode which functions as the anode and the electrode which functions as the cathode.

13. The tactile sensation presenting device of claim 11 wherein, in adjusting the execution mode of the electrical stimulation, the control section adjusts a frequency of the voltage applied to the electrode which functions as the anode and the electrode which functions as the cathode.

14. The tactile sensation presenting device of claim 1, wherein the plurality of electrodes are arrayed in a matrix including a plurality of rows and a plurality of columns.

15. The tactile sensation presenting device of claim 1, wherein
the plurality of electrodes include a plurality of anodic electrodes and a plurality of cathodic electrodes,
the control section is capable of independently switching each of the plurality of anodic electrodes between a state where the anodic electrode is supplied with an anodic potential and a state where the anodic electrode is supplied with a floating potential, and
the control section is capable of independently switching each of the plurality of cathodic electrodes between a state where the cathodic electrode is supplied with a cathodic potential and a state where the cathodic electrode is supplied with a floating potential.

16. The tactile sensation presenting device of claim 15, wherein each of the plurality of cathodic electrodes has such a shape that surrounds at least one of the plurality of anodic electrodes as viewed in plan.

17. The tactile sensation presenting device of claim 1, wherein the specific portion is a fingertip inner portion of the user.
